(19) United States
(12) Patent Application Publication
(10) Pub. No.: US 2003/0166651 A1
(43) Pub. Date: Sep. 4, 2003

(54) Novel 3-substituted 1,2,4-benzotriazines, a method for their production and the use thereof

(76) Inventors: Ulrich Stottmeister, Leipzig (DE); Manfred Schonfelder, Leipzig (DE); Horst Wilde, Leipzig (DE); Dieter Sicker, Leipzig (DE); Jens Andersch, Mittweida (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/168,346

(22) PCT Filed: Dec. 20, 2000
(86) PCT No.: PCT/EP00/13028

(30) Foreign Application Priority Data
Dec. 21, 1999 (DE) 19963316.9

(51) Int. Cl. A61K 31/5377; A61K 31/53; C07D 413/02; C07D 253/08

(52) U.S. Cl. 514/234.2; 514/243; 544/112; 544/183

(57) ABSTRACT

The invention relates to novel 3-substituted 1,2,4-benzotriazines of general formula I. The invention is also directed to a method of producing said 3-substituted 1,2,4-benzotriazines and to the use thereof in the treatment and prophylaxis of tumor-related diseases.

The invention relates to novel 3-substituted 1,2,4-benzotriazines of general formula I. The invention is also directed to a method of producing said 3-substituted 1,2,4-benzotriazines and to the use thereof in the treatment and prophylaxis of tumor-related diseases.

Compounds having anticarcinogenic activity are known in large numbers. In the class of 1,2,4-triazines, the cancerostatic effects of 3-amino- and 3-alkyl-1,2,4-benzotriazine 1,4-dioxide have been described. Thus, for example, Monge, Antonio et al., Med. Chem., EN, 38, 10, 1995, 1786-1792; and J. Med. Chem., EN, 38, 22, 1995, 4488-4494; have presented cytotoxic studies using 3-amino-1,2,4-benzotriazine 1,4-dioxide on V-79 cells (Chinese hamster lung fibroblasts). Synthesis and antitumor activity of other new analogues have been demonstrated by Tracy, M. on a Workshop held by the British Association of Cancer at Oxford, England (Sep. 17-19, 1992). The antitumor activity of 3-alkyl-1,2,4-benzotriazine 1,4-dioxides has been the subject matter on the VIII^{th} Meeting on chemical modifiers in the treatment of cancer, Kyoto, Japan, Jun. 16-19, 1993. Also, 4-amino-3-hydrazino-6-methyl-4(H)-1,2,4-triazin-5-one is known to have cancerostatic activity (Borrell, J. I. et al., ANQUEX; An. Quim., EN, 91, 3-4, 243-252, 1995).

The invention was based on the object of synthesizing further novel compounds having cytostatic activity, which compounds could be introduced in cells, inhibit proliferation of tumor cells and thus, would be suitable in the production of drugs for the treatment and prophylaxis of tumor-related diseases. Another object of the invention was to devise a method of producing said compounds, which method would be simple with respect to performing the reactions, and would provide the compounds in good yields.

Said object is accomplished according to the claims. The invention is directed to novel 3-substituted 1,2,4-benzotriazines of general formula I

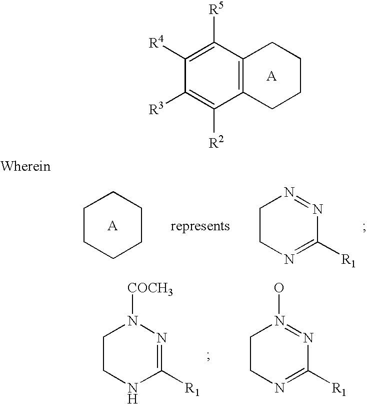

R¹ represents a straight-chain or branched, saturated or mono- or polyunsaturated hydrocarbon residue with 1 to 22 C atoms which is unsubstituted or mono- or polysubstituted by halogen, oxygen, hydroxy, amino, alkoxy, alkoxycarbonyl, morpholino and/or alkylamino, an unsubstituted or substituted aryl residue, or a heterocyclic residue, and

R², R³, R⁴, and R⁵, same or different, represent hydrogen, halogen, NO₂, NH₂, OH, or an alkoxy or alkyl residue having 1-6 C atoms.

The compounds of general formula I are biologically active, having pronounced antitumor activity. In particular, they can be used with advantage in the production of drugs for the treatment and prophylaxis of tumor-related diseases.

Preferred compounds with antitumor activity are 1,2,4 benzotriazines wherein

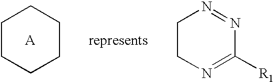

and

R¹ represents an alkyl or alkyl-COO-alkyl residue, an alkyl residue (polyolyl residue) substituted with hydroxy groups or substituted with hydroxy groups and —COO-alkyl groups, an alkyl-CO—NH-alkyl residue, or a polyolyl residue substituted with morpholin-4-yl-CO—, wherein each alkyl represents a residue having a chain length of from 1 to 6 C atoms.

Those compounds are particularly preferred wherein

R¹ represents —CH₃, —CH₂—COO—CH₃, —CH₂—COO—CH₃, —CH₂—COO—CH₃, —CH₂—COO—C₂H₅, D-arabino-(CHOH)₃—CH₂OH, D-ribo-(CHOH)₃—CH₂OH, D-galacto-(CHOH)₄—CH₂OH, D-galacto-(CHOH)₄—COO—C₂H₅, D-arabino-(CHOH)₃—CH₂OH, —CH₂—CO—NH—CH(CH₃)₂, L-arabino-

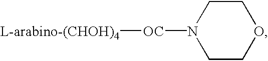

and

R², R³, R⁴, and R⁵, same or different, represent H, Cl, Br and NH₂.

Highly preferred compounds are:

a) 3-methyl-1,2,4-benzotriazine (R¹=—CH₃, R², R³, R⁴, R⁵=H),

b) 3-(methoxycarbonylmethyl)-6-chlorobenzo-1,2,4-triazine (R¹=—CH₂—CO—OCH₃, R², R³, R⁵=H, and R⁴=Cl),

c) 3-(methoxycarbonylmethyl)-5-chlorobenzo-1,2,4-triazine (R¹=—CH₂—CO—OCH₃, R², R⁴, R⁵=H, and R³=Cl),

d) 3-(methoxycarbonylmethyl)-5-amino-6-chlorobenzo-1,2,4-triazine (R¹=—CH₂—CO—OCH₃, R³=NH₂, R⁴=Cl, R², R⁵=H),

e) 3-(methoxycarbonylmethyl)-1,2,4-benzotriazine (R¹=—CH₂—CO—OCH₃, R², R³, R⁴, R⁵=H),

f) 3-(ethoxycarbonylmethyl)-1,2,4-benzotriazine (R¹=—CH₂—CO—OC₂H₅, R², R³, R⁴, R⁵=H),

g) 3-(ethoxycarbonylmethyl)-6-bromobenzo-1,2,4-triazine (R¹=—CH₂—CO—OC₂H₅, R², R³, R⁵=H, and R⁴=Br),

h) 3-(D-arabino-tetritol-1-yl)-1,2,4-benzotriazine (R¹=D-arabino-(CHOH)₃—CH₂OH, R², R³, R⁴, R⁵=H),

i) 3-(D-galacto-tetritol-1-yl)-1,2,4-benzotriazine (R¹=D-galacto-(CHOH)₄—CH₂OH, R², R³, R⁴, R⁵=H),

j) 3-(ethoxycarbonyl-D-galacto-tetritol-1-yl)-1,2,4-benzotriazine (R¹=D-galacto-(CHOH)₄—CO—OC₂H₅, R², R³, R⁴, R⁵=H),

k) 3-(D-arabino-tetritol-1-yl)-6-chlorobenzo-1,2,4-triazine (R¹=D-arabino-(CHOH)₃—CH₂OH, R², R³, R⁵=H, R⁴=Cl),

l) 3-(isopropylaminocarbonylmethyl)-1,2,4-benzotriazine (R¹=—CH₂—CO—NH—CH(CH₃)₂, R², R³, R⁴, R⁵=H),

m) 3-(D-ribo-tetritol-1-yl)-1,2,4-benzotriazine (R¹=D-ribo-(CHOH)₃—CH₂OH, R², R³, R⁴, R⁵=H),

n) 3-(morpholin-4-ylcarbonyl-L-arabino-tetritol-1-yl)-1,2,4-benzotriazine (R¹=L-arabino-(CHOH)₄—

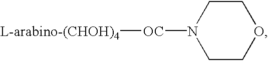

R², R³, R⁴, R⁵=H).

These new compounds of general formula I can be synthesized according to per se known methods (e.g., Jerschel, D. and Edler, W., Chem. Ber. 88, 1284 (1955); Fusco, R. and Bianchetti, G., Gazz. Chim. Ital. 90, 1113 (1960); or Kwee, S. and Lund, H., Acta Chem. Scand. 23, 2711 (1969)), by reducing the 2-nitrophenylhydrazones of carbonyl compounds or the tautomeric azo compounds.

According to the invention, the compounds are obtained by reacting an appropriate 2-nitrophenylhydrazine with an appropriate 2-oxocarboxylic acid or a salt thereof according to the scheme below to form the 2-nitrophenylhydrazone. The 2-nitrophenylhydrazone having formed is subsequently reduced catalytically using hydrogen to yield the target compound of general formula I.

Scheme:

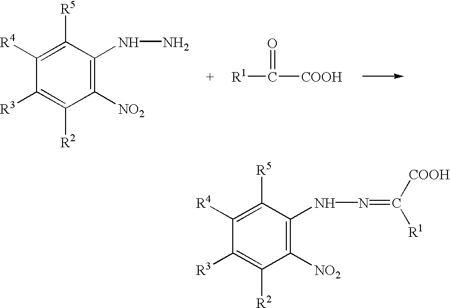

wherein R¹ through R⁵ have the above-mentioned meanings.

In another embodiment of the invention, the 1,4-dihydro-1,2,4-benzotriazines formed as intermediates are isolated by filtrating the 1,4-dihydro-1,2,4-benzotriazine from the catalyst under an inert gas atmosphere after completed hydrogen uptake and distilling off the solvent. Acetylation using acetic anhydride, preferably at 20° C., furnishes the monoacetyl derivatives of general formula I.

Preferred monoacetyl compounds with antitumor activity are those wherein

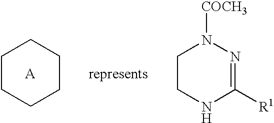

and R¹ represents —CH₂—CO—OC₂H₅. Particularly preferred is compound o) with R², R³, R⁴, R⁵=H.

The substituted or unsubstituted 2-nitrophenylhydrazines employed are well-known per se, being prepared by reducing diazonium compounds (Mangini, Deliddo, Gazz. Chim. Ital. 63, 612, 1933) or by reacting 2-nitrohalobenzenes with hydrazine hydrate (Albini, Angelo et al., Heterocycles 597, 1995).

In a preferred embodiment of said method, fermentation solutions including the corresponding 2-oxocarboxylic acid are used directly in the synthesis of said 2-nitrophenylhydrazones. Preferably, this is the case with fermentation solutions including 2-oxoglutaric acid or 2-oxogluconic acid.

As a rule, the content of 2-oxocarboxylic acid required for reaction in the fermentation solution is about 15%, preferably >20%.

The 2-nitrophenylhydrazine to be reacted is dissolved in an organic solvent or mixture of solvents, optionally with heating, and added at a temperature of from 20 to 80° C., preferably 45-55° C., to a fermentation solution including the corresponding 2-oxocarboxylic acid. The 2-nitrophenylhydrazone having formed is subjected to a secondary treatment, if required. The target compounds of general formula I are obtained in high yields of >30%, preferably >50%, by catalytic reduction with subsequent cyclization, decarboxylation and oxidation.

This method is highly advantageous, i.e., it can be performed in a one-pot reaction and therefore, with respect to conducting the reaction and with respect to the yields achieved, is superior to the well-known paths of synthesis where yields normally are below 30%.

Furthermore, compounds of general formula I with antitumor activity preferably are 1,2,4-benzotriazines wherein

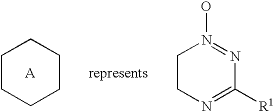

and R¹ represents NH₂. Compound p) with R², R₃, R⁴, R⁵=H was found to be particularly effective.

These compounds are produced using per se known methods according to Suzuki, H. and Kawakami, T., Synthesis 1997, 855-857. 2-Fluoronitrobenzene and guanidine are stirred in THF at 60° C. for about 4 hours, followed by addition of tert-BuOK and stirring for another 2 hours. The precipitated product is isolated.

With reference to the examples, the invention will be illustrated in more detail below.

### EXAMPLE 1

### 3-Methyl-1,2,4-benzotriazines

0.01 mol of sodium pyruvate and 0.01 mol of the corresponding 2-nitrophenylhydrazine are reacted with stirring in a solution of 10 ml of ethanol and 10 ml of 35% perchloric acid. The yellow precipitate obtained after a short time is sucked off, washed with water to neutrality, dried and subsequently recrystallized from ethanol.

Yield: 90-95% of theoretical amount.

3 mmol of the respective 2-nitrophenylhydrazone thus obtained is dissolved in 50 ml of ethanol and hydrogenated over platinum oxide at room temperature and normal pressure until the hydrogen absorption ceases. Thereafter, the catalyst is filtrated off and the solvent is distilled off under vacuum. Each of the remaining 3-methyl-1,2,4-benzotriazines is recrystallized from petroleum ether.

Yield: 55-70% of theoretical amount.

### EXAMPLE 2

### 3-(D-arabino-Tetritol-1-yl)-1,2,4-benzotriazine

10 mmol of 2-nitrophenylhydrazine is dissolved in 30 ml of methanol and 0.5 ml of acetic acid with warming. The solution is added dropwise to a fermentation solution at 50° C. with stirring, which has a 24% content of 2-oxo-D-gluconic acid. The reaction mixture is stirred for one more hour. The orange precipitate is sucked off, washed, and recrystallized from water.

Yield: 90% of theoretical amount.

A suspension of 6 mmol of the thus obtained 2-nitrophenyl hydrazone in 60 ml of tetrahydrofuran is hydrogenated over Pt/C under normal pressure at room temperature until the calculated hydrogen absorption is reached. The reaction mixture is stirred for 3 days under aerobic conditions. Thereafter, the solvent is removed, and the product is separated from the catalyst by extraction with methanol.

Yield: 30% of theoretical amount; yellow crystals, m.p.: 173-175° C.

### EXAMPLE 3

### Inhibition of Cell Growth by the Compounds of the Invention

Aqueous sterile solutions of each substance to be tested are injected into 1 ml of the respective cell stock solution at concentration steps of 2-50 μg each time. Subsequently, the test chambers are developed together with a non-treated reference chamber for three days at 37° C. Assessment is performed using a microscope. Cell growth or cell decomposition (condition of cell membrane and cell nucleus) is compared to non-treated reference cells.

TABLE
IC₅₀ value (50% inhibition of cell growth in μg/ml) of
selected substances of formula I

| | | | Peritoneal | Pleural |
|---|---|---|---|---|
| Substance | Hep2 | HeLa | carcinoma | carcinoma |
| Compound b | 120 | 160 | 150 | 500 |
| Compound c | 16 | 8-16 | 12 | |
| Compound d | 500 | 50 | 25-30 | 500 |
| Compound e | 100 | 180 | 350 | 500 |
| Compound f | 190-200 | 378 | 180 | 500 |
| Compound g | 8-16 | 8-16 | 35 | |
| Compound h | 150 | 150 | | |
| Compound i | | 400 | | |
| Compound j | | | | |
| Compound k | | 250 | | |
| Compound l | 100 | 86 | 170 (100%) | |
| Compound o | 12 | 12 | | |
| Compound p | 25 | 30 | | |

All of the compounds according to the invention inhibit cell growth of permanent cell cultures such as HeLa, Hep2 cells, of peritoneal carcinoma and pleural carcinoma cells.

As can be seen from the Table above, all of the compounds are effective, and the compounds c, g, l, o, and p have preferred activity.

Selective in vitro antitumor activity has been found, i.e., the growth of normal cells within a comparable concentration range is inhibited to a much lesser extent than tumor growth.

1. Novel 3-substituted 1,2,4-benzotriazines of general formula I 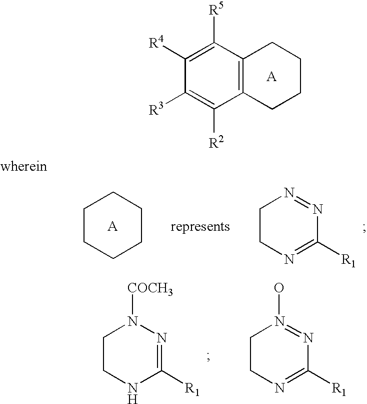
R¹ represents a straight-chain or branched, saturated or mono- or polyunsaturated hydrocarbon residue with 1 to 22 C atoms which is unsubstituted or mono- or polysubstituted by halogen, oxygen, hydroxy, amino, alkoxy, alkoxycarbonyl, morpholino and/or alkylamino, an unsubstituted or substituted aryl residue, or a heterocyclic residue, and
R², R³, R⁴, and R⁵, same or different, represent hydrogen, halogen, NO₂, NH₂, OH, or an alkoxy or alkyl residue having 1-6 C atoms.

2. The 1,2,4-benzotriazines according to claim 1, characterized in that 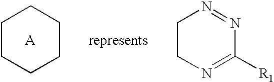
and
R¹ represents an alkyl or alkyl-COO-alkyl residue, an alkyl residue (polyolyl residue) substituted with hydroxy groups or substituted with hydroxy groups and —COO-alkyl groups, an alkyl-CO—NH-alkyl residue, or a polyolyl residue substituted with morpholin-4-yl-CO—, wherein each alkyl represents a residue having a chain length of from 1 to 6 C atoms.

3. The 1,2,4-benzotriazines according to claim 1 or 2, characterized in that
R¹ is —CH₃, —CH₂—COO—CH₃, —CH₂—COO—CH₃, —CH₂—COO—CH₃, —CH₂—COO—C₂H₅, D-arabino-(CHOH)₃—CH₂OH, D-ribo-(CHOH)₃—CH₂OH, D-galacto-(CHOH)₄—CH₂OH, D-galacto-(CHOH)₄—COO—C₂H₅, D-arabino-(CHOH)₃—CH₂OH, —CH₂—CO—NH—CH(CH₃)₂, L-arabino-(CHOH)

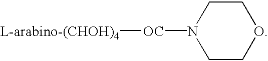

4. The 1,2,4-benzotriazines according to claim 3, characterized in that
R², R³, R⁴, and R⁵, same or different, represent H, Cl, Br, NH₂.

5. The 1,2,4-benzotriazines according to claim 1, characterized in that 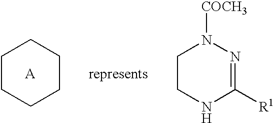
and
R¹ represents —CH₂—CO—OC₂H₅.

6. The 1,2,4-benzotriazines according to claim 5, characterized in that
R², R³, R₄, R⁵ represent hydrogen.

7. The 1,2,4-benzotriazines according to claim 1, characterized in that 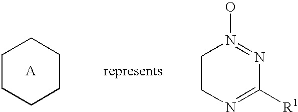
and
R¹ represents NH₂.

8. The 1,2,4-benzotriazines according to claim 7, characterized in that
R², R³, R⁴, R⁵ represent hydrogen.

9. A method of producing a 3-substituted 1,2,4-benzotriazine of general formula I according to any of claims 1 to 4, characterized in that
an appropriate 2-nitrophenylhydrazine is reacted with an appropriate 2-oxocarboxylic acid to form the 2-nitrophenylhydrazone, and that the 2-nitrophenylhydrazone obtained is subsequently reduced catalytically using hydrogen to yield the target compound of general formula I.

10. A method of producing a 3-substituted 1,2,4-benzotriazine of general formula I according to any of claims 1, 5 to 6, characterized in that
an appropriate 2-nitrophenylhydrazine is reacted with an appropriate 2-oxocarboxylic acid to form the 2-nitrophenylhydrazone, and that the 2-nitrophenylhydrazone obtained is subsequently reduced catalytically using hydrogen, the 1,4-dihydro-1,2,4-benzotriazine formed as intermediate is isolated after completed hydrogen uptake by filtrating from the catalyst under an inert gas atmosphere, the solvent is distilled off, and an acetylation is performed.

11. The method according to any of claims 9 to 10, characterized in that
the 2-nitrophenylhydrazine is reacted with a fermentation solution including a 2-oxocarboxylic acid.

12. The method according to claim 11, characterized in that
the fermentation solution includes the 2-oxocarboxylic acid with a content of at least 10%, preferably >20%.

13. The method according to claim 11 or 12, characterized in that
the reaction to form the target compound I is performed in the same reaction vessel without previous isolation of the 2-nitrophenylhydrazone.

14. A method of producing a 3-substituted 1,2,4-benzotriazine of general formula I according to any of claims 1, 7 to 8, characterized in that
2-fluorobenzene and guanidine are reacted in THF, tert-BuOK is subsequently added, and the precipitated N-oxide is isolated.

15. Use of 3-substituted 1,2,4-benzotriazines of general formula I according to any of claims 1 to 8 in the production of drugs for the treatment and prophylaxis of tumor-related diseases.
